(19) United States
(12) Patent Application Publication
(10) Pub. No.: US 2021/0219842 A1
(43) Pub. Date: Jul. 22, 2021

(54) ILLUMINATOR WITH MAGNIFICATION AND MULTIPLE LIGHTING MODES

(71) Applicant: 3GEN, INC., San Juan Capistrano, CA (US)

(72) Inventors: Thorsten Trotzenberg, Wiesbaden (DE); Gregory Paul Lozano-Buhl, Grand Rapids, MI (US)

(73) Assignee: 3GEN, INC., San Juan Capistrano, CA (US)

(21) Appl. No.: 17/153,640

(22) Filed: Jan. 20, 2021

Related U.S. Application Data
(60) Provisional application No. 62/970,643, filed on Feb. 5, 2020.
(60) Provisional application No. 62/964,425, filed on Jan. 22, 2020.

(51) Int. Cl. A61B 5/00; F21V 9/06; F21V 9/14; F21V 23/04; G02B 25/00; G02B 3/04; A61B 90/00

(52) U.S. Cl. CPC A61B 5/0077; F21V 9/06; F21V 9/14; F21V 23/04; F21Y 2115/10; G02B 3/04; G02B 25/008; A61B 90/36; G02B 25/002

(57) ABSTRACT

The illuminator device for medical examination described herein employs a housing incorporating a magnified viewing lens having a lens polarizer and an array of LEDs to provide light for the viewing organic tissue and other matter. A switch is provided to communicate with a microprocessor that controls an LED driver adapted initiate to provide modes of operation that provide certain of the LEDs being illuminated in different modes. In operation the device incorporates at least five modes of operation. A first mode provides activating only polarized white lights, a second mode provides activating only ZWB2 bandpass filtered 365 nm UV LEDs, a third mode provides activating only ZWB2 bandpass filtered 385 nm UV LEDs, a fourth mode provides activating only unfiltered 405 nm UV LEDs and a fifth mode comprises activating only ZWB2 bandpass filtered 365 nm UV LEDs and ZWB2 bandpass filtered 385 nm UV LEDs. The device additionally incorporates an auxiliary magnifier that stores in the device handle.

### CROSS-REFERENCE TO RELATED APPLICATIONS

The present application claims the benefit of U.S. Provisional Application No. 62/964,425, filed Jan. 22, 2020, and U.S. Provisional Application No. 62/970,643, filed Feb. 5, 2020, the contents of each of which are expressly incorporated herein by reference in their entireties.

### STATEMENT RE: FEDERALLY SPONSORED RESEARCH/DEVELOPMENT

Not Applicable

### BACKGROUND

### Technical Field

The present inventive subject matter relates generally to a hand-held illumination device used in medical examinations. More particularly, the inventive subject matter relates to an improved apparatus for viewing and illumination of the skin for medical examinations using magnifiers with arrangements of light emitting diodes that operate in multiple lighting modes.

### Background

Medical examinations by physicians and healthcare providers may employ the use of hand-held illuminators to assist the doctor in magnified and non-magnified viewing of the tissue of a patient. Hand-held illuminators without magnification include pen lights, which are widely used by general medical practitioners. Also, physicians and medical practitioners make use of hand-held illumination devices that have magnification including otoscopes, ophthalmoscopes and dermatoscopes. Otoscopes, ophthalmoscope and dermatoscopes include a lens for magnification and may have single or multiple lighting modes. Also, standard otoscopes and ophthalmoscope may not employ polarization of light and viewing lenses.

Hand-held dermoscopy devices that use light with magnification can utilize polarizers or liquid-glass interfaces to reduce surface reflection and aid in viewing of deeper structures in the skin. Dermoscopy apparatuses that employ light polarization to aid in viewing human skin surfaces and deeper tissue and structures of the skin are known and described U.S. Pat. No. 7,006,223 entitled Dermoscopy epiluminescence device employing cross and parallel polarization issued on Feb. 28, 2006 to Mullani, and U.S. Pat. No. 7,167,243 entitled Dermoscopy epiluminescence device employing cross and parallel polarization issued Jan. 23, 2007 to Mullani, the substance of each of which is wholly incorporated herein by reference. In addition, a dermoscopy device identified as Dermlite® DL3 device is manufactured and marketed by 3Gen, Inc. of San Juan Capistrano which uses light and polarization. In the Dermlite® DL3 hand held device, a series of light emitting diodes (“LEDs”) are concentrically positioned around a magnifying lens to assist in lighting of a magnified image. The device includes LEDs that provide reduced glare, cross-polarized light to aid in canceling the reflected light from the skin, and other LEDs on the device provide non-polarized light for traditional immersion fluid dermoscopy or for simply employing non-polarized light.

It is also well known that different colored light penetrates to different depths in human skin tissue. Specific color wavelengths are absorbed differently by different components of the skin tissue. Such use of colored LEDs in a dermatoscope is described in U.S. Pat. No. 7,027,153 entitled Epiluminescence Device Employing Multiple Color Illumination Sources issued on Apr. 11, 2006 to Mullani and U.S. Pat. No. 7,167,244 entitled Epiluminescence Device Employing Multiple Color Illumination Sources issued on Jan. 23, 2007 to Mullani, the substance of each of which is wholly incorporated herein by reference. The previously identified references disclose the combined use of white LEDs, UV/blue LEDs (405 nm), green/yellow LEDs (565 nm) and orange/red (630 nm). Alternatively, the U.S. Pat. Nos. 7,027,153 and 7,167,244 references suggest the use of LEDs with 480 nm, 580 nm and 660 nm wavelengths. In addition, a dermoscopy device identified as Dermlite® II Multispectral dermoscopy device manufactured and marketed by 3Gen, Inc. of San Juan Capistrano, Calif. provides four sets of LED's comprising white, blue light (470 nm) for surface pigmentation, yellow light (580 nm) for superficial vascularity viewing, and red light (660 nm) for viewing of pigmentation and vascularity with the deeper-penetrating red light frequency.

Dermatoscopes using coloured LEDS to augment the viewing of pigmentation of human tissue including skin is shown and described in U.S. Pat. No. 9,458,990 entitled Dermoscopy Illumination Device with Selective Polarization and Orange Light For Enhanced Viewing of Pigmented Tissue issued Oct. 4, 2016 to Mullani the substance of which is wholly incorporated herein by reference. In addition, a dermoscopy device identified as Dermlite® DL4 dermoscopy device manufactured and marketed by 3Gen, Inc. of San Juan Capistrano, Calif. provides combinations of white LED lights and orange LED lights in both polarized and non-polarized combinations to provide enhanced viewing of skin pigmentation.

Furthermore, hand held medical illuminators have been used to introduce light into human tissue for observing sub-dermal structures using side-transillumination techniques whereby the light source is caused to be in direct contact with the skin to transfer light directly into the skin. One such technique is known and taught in U.S. Pat. No. 5,146,923 entitled Apparatus and Method for Skin Lesion Examination issued on Sep. 15, 1992 to Dhawan, the substance of which is wholly incorporated herein by reference. A combination of surface illumination, epiluminescence and transillumination apparatus and method is demonstrated in the Nevoscope™ product sold manufactured by Translite LLC, of Sugar Land, Tex. Another known apparatus and method of viewing vein structures beneath the skin employs the use of transillumination as described in U.S. Pat. No. 7,874,698 entitled Transillumination Having Orange Color Light issued on Jan. 25, 2011 to Mullani the substance of which is wholly incorporated herein by reference. U.S. Pat. No. 7,874,698 issued to Mullani describes the use of orange light between 580 and 620 nm for transillumination imaging of deeper blood vessels in skin tissue.

Wide lens dermatoscopes have been used to provide a wider area of view for use in skin examination. For example, a wide lens dermatoscope identified as Lumio® manufactured and marketed by 3Gen, Inc. of San Juan Capistrano, Calif. is used for general skin exams to provide reduced glare viewing through a wide magnification lens (75 mm diameter) at 2× magnification and 40 bright white LEDs employing cross polarization. A sister product Lumio® UV manufactured and marketed by 3Gen, Inc. of San Juan Capistrano, Calif. also employs a 2× magnification 75 mm diameter lens, with UV light emitted from 40 UV LEDs. Also, the use of magnets in coupling two optical devices together using magnets placed in anti-parallel arrangement for functional use is shown and described in U.S. Pat. No. 10,678,120 entitled Medical Illuminator Mobile Device Attachment Apparatus and Method issued on Jun. 9, 2020 to Lozano-Buhl et al. and United States Patent Application Publication 2020/0237310 entitled Medical Illuminator Mobile Device Attachment Apparatus and Method published Jul. 30, 2020 to Lozano-Buhl et al, the substance of each of which are incorporated herein by reference in their entireties.

There is a need in the art for a wide lens dermatoscope that includes multiple lighting modes in a single device and also provides glare reduction through use of polarization and cross polarization.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### BRIEF SUMMARY

The inventive subject matter described herein demonstrates an illuminator used in medical examinations. The illuminator device described herein employs a housing incorporating a magnification viewing lenses that allows medical practitioners to view patient tissue and structures with a wider viewing angle than prior dermatoscopes that deploy multiple types of LEDs and lighting modes. A battery power source is contained in the housing to provide power to an illumination source. An illumination source may include an array of LEDs to provide light for viewing the patient tissue and other matter. A switch is provided to initiate the LED illumination source or to provide modes of operation that provide certain of the LEDs being illuminated.

The illuminator device described herein employs a housing incorporating a magnified viewing lens having a lens polarizer and an array of LEDs to provide light for the viewing organic tissue and other matter. A switch is provided to communicate with a microprocessor that controls an LED driver adapted to initiate modes of operation that provide certain of the LEDs being illuminated in different lighting modes. Certain of the LEDs may include a bandpass filter placed over such LEDs to filter the light emitted from the LEDs. The utilized bandpass filter for a particular LED attenuates all or a significant portion of frequencies outside of a desired frequency. Bandpass filters are placed over the light transmitting LED and may transmit a desired frequency range through the filter, while blocking all or most of certain frequency ranges of light from passing through the filter. In an embodiment of the device, a ZWB2 bandpass filter is used. Use of ZWB2 bandpass filters result in much better contrast in an image due to the elimination of all or a significant portion of visible light. Although an embodiment of the device discloses a ZWB2 filter, it is contemplated by the present disclosure that other bandpass filters may be utilized that have a similar effect of a ZWB2 namely an optical filter that has higher transmittance in the UV spectrum than the visible spectrum. In operation, an embodiment of the device incorporates at least five modes of operation. A first mode provides activating only polarized white lights, a second mode provides activating only ZWB2 bandpass filtered 365 nm UV LEDs, a third mode provides activating only ZWB2 bandpass filtered 385 nm UV LEDs, a fourth mode provides activating only unfiltered 405 nm UV LEDs and a fifth mode (Woods mode) comprises activating only ZWB2 bandpass filtered 365 nm UV LEDs and ZWB2 bandpass filtered 385 nm UV LEDs. LEDs are formed on a conically configured LED PCB and is situated in the housing to provide the LEDs at angled placement about the circumference of magnified viewing lens. The device additionally incorporates an auxiliary magnifier that stores in the device handle when not in use. In operation, the auxiliary magnifier may be removed from the handle and attached to the device, via magnets, to the viewing side of the illuminator device, with the magnifier placed over at least a portion of the magnifying lens to provide enhanced view of an area of concern without the need to use a different dermatoscope. Magnets situated in the device are axially magnetized and placed in antiparallel relationship to each other, and corresponding magnets attached in auxiliary magnifier, are also axially magnetized and interact with the magnets of the device for placement on the device. Similarly, the device includes an auxiliary bandpass filter, that may also be stored in the device handle. In operation, the auxiliary bandpass filter may be attached to the device, via magnets, to the viewing side of the illuminator device, with the bandpass filter placed over at least a portion of the magnifying lens to provide a further filtered view of an area of concern. Corresponding magnets attached in the auxiliary bandpass filter, are axially magnetized, are axially magnetized and interact with the magnets of the device for placement on the device.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a distal upper view of the disclosed dermatoscope device;

FIG. 2 is right side view of the disclosed dermatoscope device;

FIG. 3 is a proximal bottom view of the disclosed dermatoscope device;

FIG. 4 is a cross sectional view of disclosed device the along the axis 4-4 as shown in FIG. 1;

FIG. 5 is a cross sectional view of the disclosed device along the axis 5-5 as shown in FIG. 1;

FIG. 6 is an exploded view of the disclosed device showing various components of the disclosed device;

FIG. 7 a plan view of the upper body component of the disclosed device;

FIG. 8 side perspective view of the upper body component of the disclosed device showing magnets exploded from the component;

FIG. 9 is a plan view of upper body component of the disclosed device with a nested PCB board containing LEDs and electronic components with electronic leads and a power port interface;

FIG. 10 is a side perspective view of the upper body component of the device of the disclosed device with the PCB board containing LEDs and electronic components, electronic leads and power port interface exploded from the upper body component;

FIG. 11 is a reverse view of the PCB board containing electronic components, a reverse view of the PCB board containing LEDS, electronic leads and power port interface;

FIG. 12 is side perspective view of the LED PCB board;

FIG. 13 is front plan view of the LED PCB board;

FIG. 14 is a rear view of the LED PCB board;

FIG. 14A is a perspective view of the LED PCB board with ends attached in an assembled configuration;

FIG. 14B is a top plan view of the LED PCB board with ends attached in an assembled configuration;

FIG. 14C is cross sectional view of the LED PCB board along axis 14C-14C as shown in FIG. 14B;

FIG. 14D is a cross sectional view of the LED PCB board demonstrating the angled placement of the PCB bard in an assembled configuration;

FIG. 15 is a plan view of upper body component of the disclosed device including the nested components of FIG. 9, overlaid with a light separator component and battery;

FIG. 16 is a side perspective view of the upper body component of the disclosed device including the nested components of FIG. 9, with the light separator component and battery exploded from the upper body component;

FIG. 17 is a plan view of the upper body component of the disclosed device including the nested components of FIG. 9, including a light separator component and battery incorporating ZWB2 filters placed over certain of the LEDs of the device;

FIG. 18 is a side perspective view of the upper body component of the disclosed device including the nested components of FIG. 9, including a light separator component and battery with the ZWB2 filters exploded from the device showing placement;

FIG. 19 is a perspective view of the upper body component of the disclosed device including the nested components of FIG. 9, including a light separator component and battery incorporating ZWB2 filters, incorporating a further LED ring filter;

FIG. 20 is a perspective view of the upper body component of the disclosed device including the nested components of FIG. 9, including a light separator component and battery incorporating ZWB2 filters as shown in FIG. 17, with the LED ring filter exploded showing placement;

FIG. 21 is a plan view of the viewing region of the disclosed device showing LED placement, ZWB2 filter placement and ring filter placement;

FIG. 22 shows a perspective view of the disclosed device showing placement for use of a nesting auxiliary magnifier;

FIG. 23 shows a perspective view of the disclosed device showing the nesting auxiliary magnifier exploded from the disclosed device showing placement;

FIG. 24 shows the disclosed device with metal contacts exploded from a nesting recess, with the auxiliary magnifier aligned with exploding lines;

FIG. 25 is a perspective view of the auxiliary magnifier of the disclosed device;

FIG. 26 is an exploded view of the auxiliary magnifier of the disclosed device showing components;

FIG. 27 is a perspective view of the disclosed device auxiliary magnifier, with the magnetic connection side;

FIG. 28 is a cross sectional view of the disclosed device auxiliary magnifier;

FIG. 29 is a cross sectional view of an auxiliary bandpass filter with a magnetic connection;

FIG. 30 is a schematic diagram of the electronic components of the disclosed illumination device;

FIG. 31 is a tabular chart showing transmissivity verses wavelength for ZWB2 filter glass bandpass filter; and

FIG. 32 is a graphical chart showing transmissivity verses wavelength for ZWB2 filter glass bandpass filter.

### DETAILED DESCRIPTION

The detailed description set forth below in connection with the appended drawings is intended as a description of certain embodiments of an illumination device and is not intended to represent the only forms that may be developed or utilized. The description sets forth the various structure and/or functions in connection with the illustrated embodiments, but it is to be understood, however, that the same or equivalent structure and/or functions may be accomplished by different embodiments that are also intended to be encompassed within the scope of the present disclosure. It is further understood that the use of relational terms such as first and second, and the like are used solely to distinguish one entity from another without necessarily requiring or implying any actual such relationship or order between such entities.

The background, summary and following description includes information that may be useful in understanding the present disclosure. It is not an admission that any of the information provided herein is prior art or relevant to the presently claimed inventive subject matter, or that any publication specifically or implicitly referenced is prior art.

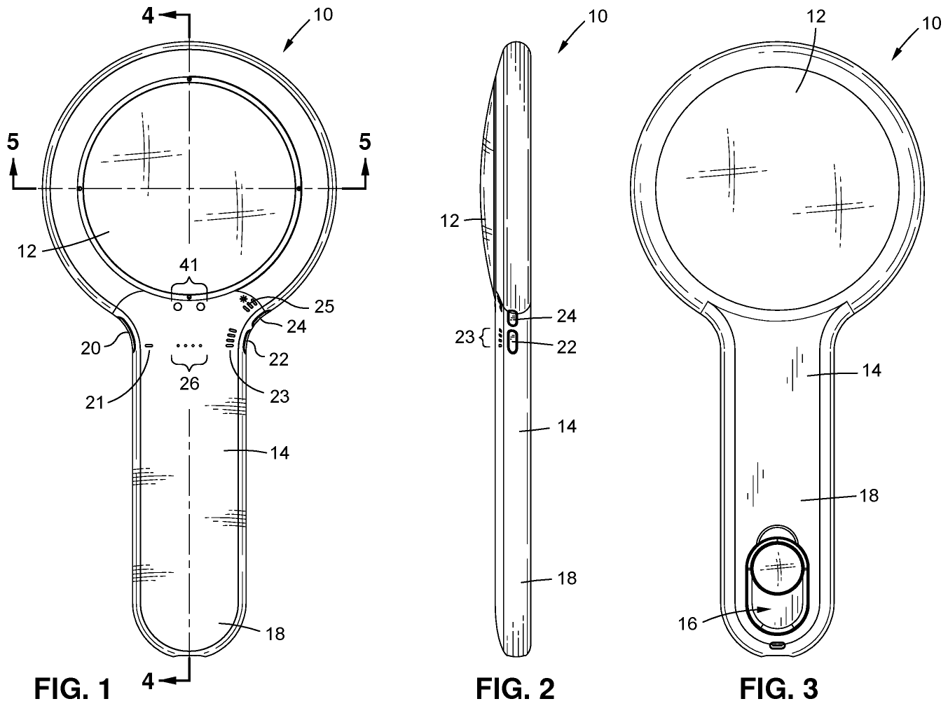

Referring particularly to FIGS. 1-3, there is shown the illumination device 10. FIG. 1 shows the distal (away from the organic material to be viewed) upper view of the illumination device, the side of the device from which a user would view through the magnification lens 12. FIG. 2 shows a right-side view of the illumination device and FIG. 3 shows the proximal (closest to the organic material to be viewed) lower view of the illumination device, the side of the device from which the LEDs emit light to the object to be viewed. FIG. 3 also shows the auxiliary or add on lens 16 removably attached to the housing 14, in particular the housing 14 handle 18.

FIGS. 1 and 2 show integrated control switches 20, 22 and 24. In operation, power switch 20 operates as follows: a single push turns on all of the white LEDs in the LED array (eight white LEDs). A white light indicator LED 21 on the handle will illuminate to indicate white light mode. A second push of power switch 20 turns on UV mode, described in more detail below. In UV mode, the user may address UV mode switch 22 to select between UV modes. There are four UV modes for the user to select by pressing the UV mode switch. UV mode indicator LEDs 23 are positioned on the handle 18 to provide an indication to the user the particular UV mode. For example, the user may select between 365 nm LEDs (four in the array), 385 nm LEDs (four in the array), 405 nm LEDs (four in the array) or Woods mode (combination of all 365 nm LEDS and 385 nm LEDS in the array). It is understood and contemplated that Woods mode could incorporate any other combinations of UV LEDs to best approximate the spectrum of a Woods Lamp. Brightness switch 24, allows the user to select between three levels of brightness. A brightness level indicator LEDs 25 provide an indication to the user of the brightness level selected. It is understood and contemplated by this disclosure that there could be any number of levels of brightness. Also, the face of device 10 also has power level indicator LEDs 26, that act as a fuel gauge indicator with all four indicators illuminated means the batter is full or nearly full. The persistent illumination (while in use) of the LED indicators 26 allows for constant monitoring of the battery level.

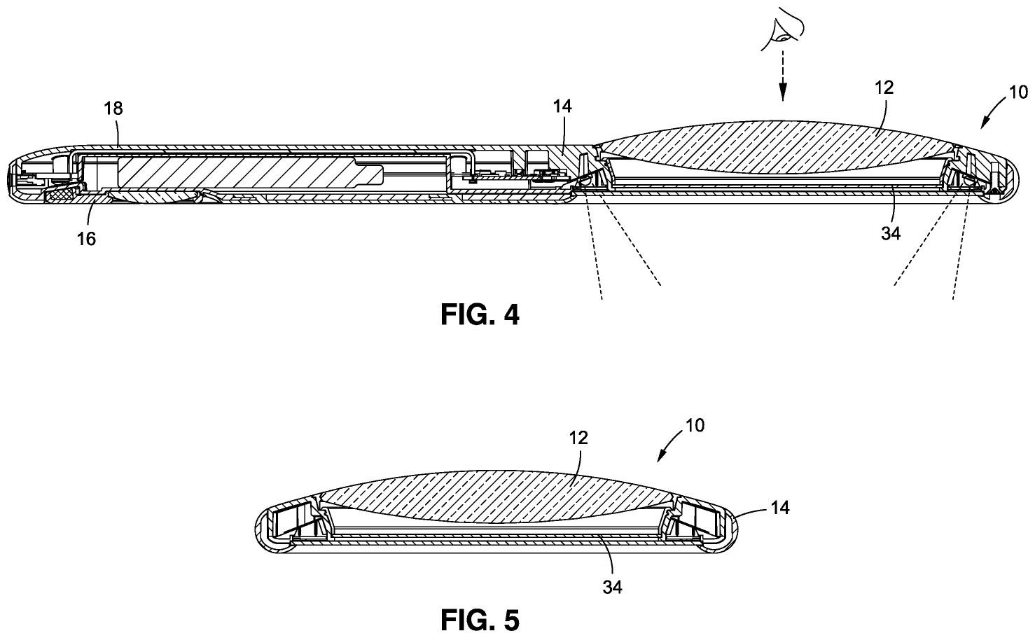

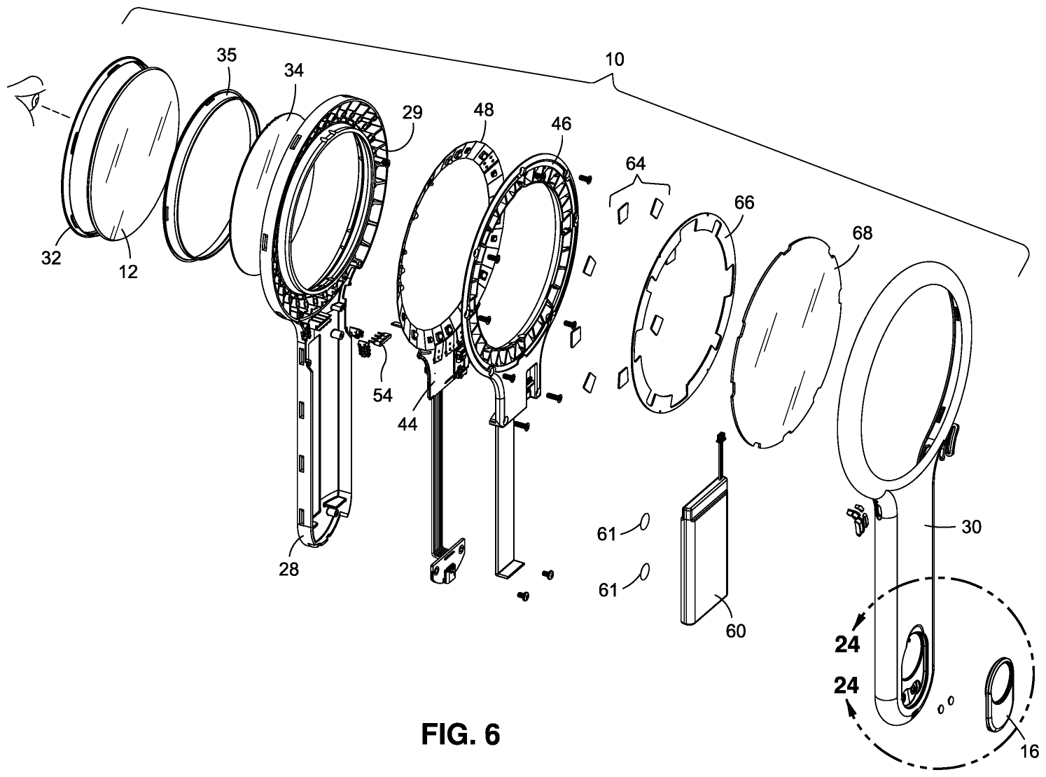

Referring particularly to FIGS. 4-6 there is shown the assembly elements of the illumination device. In particular FIG. 4 shows the cross section of the device 10 along axis 4-4 as shown in FIG. 1 and FIG. 5 shows the cross section of the device 10 along axis 5-5 in FIG. 1. FIG. 6 shows an exploded view of various elements of the device 10. FIG. 6 shows the upper body 28 and lower body 30 that collectively form the housing 14 (see FIG. 1). The housing 14 may be formed of aluminum for enhanced heat dissipation. However, it is understood that the housing 14 may be formed of any suitable rigid material such as plastic. FIG. 6 shows that a frame 32 surrounds the lens 12 to snap fit hold the lens 12 in place in the upper body 28. The lens 12 may be 100 mm in diameter, with 190 mm focal length and 2.3× magnification. The larger diameter gives a larger field of view and the lower magnification may provide ease of use, being more tolerant of varying lens-object or lens-eye distances. The lens 12 surface may have aspheric lens surfaces to help eliminate many optical aberrations such as chromatic aberration and distortion, resulting in consistent sharp image across a full field of view. As is known with spherical lenses, images may be compromised at the edges of the view. While the device disclosed herein contemplates use of an aspheric lens, other types of lenses, such as spherical or layered lens arrangements may be employed. The lens 12 material may be molded PMMA with a hard coat surface. The snap fit frame 32 allows for the user to replace the lens 12 if the lens is scratched or otherwise damaged. Other suitable material may also be used for the lens 12.

As shown in FIG. 6 a lens polarizer filter 34 is positioned in front of the lens 12 and positioned in place via a polarizer spacer 36. The spacer 36 provide positioning of the polarizer 34, via a snap frame. The polarizer filter 34 may be comprised of a linear polarized film having approximately 0.030″ thickness. Although the positioning of the axis of polarization may vary, in the disclosed device the axis of polarization is 90 degrees (perpendicular) to the axis of polarization of white light LED polarizer (described herein in greater detail). The positioning of the polarizing filter 34 is provided so that any light that is received into the lens 12 from observed tissue will pass through the filter 34 before reaching the eye of the user.

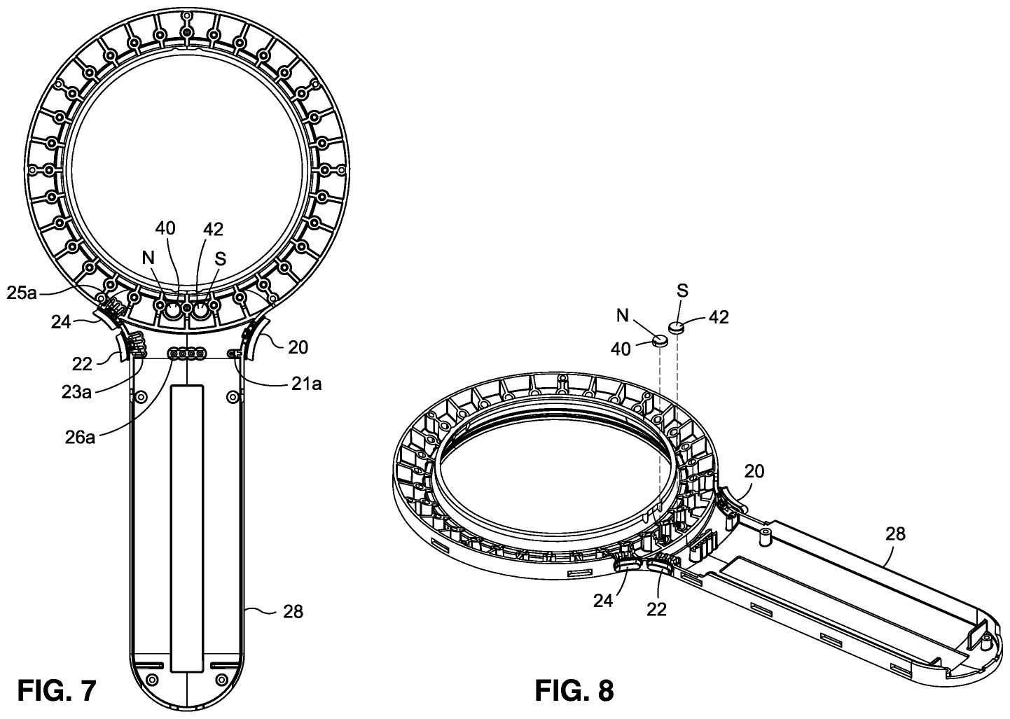

FIGS. 7 and 8 show a more detailed view of upper body 28, showing the interior of the front face of the housing 14 shown in FIG. 1. Switches 20, 22 and 24 are shown, each adjacent openings 21a, 23a, 25a and 26a that will receive indicator LEDs 21, 23, 25 and 26 upon engagement with a PCB board having such LEDs. FIGS. 7 and 8 show placement of axially magnetized magnets 40 and 42 attached to the upper body 28. Magnet positioning indicators 41 appear on the opposite front face of the body 28 as shown in FIGS. 1 and 23 to act as a guide for directing the location of the magnets and positioning an accessory magnifier in proper orientation (discussed below). The magnets 40 and 42 are positioned in antiparallel relationship to each other to provide that the accessory (also having similarly opposite polarity magnets) can be positioned in proper alignment. Likewise, use of magnets in antiparallel relationships also have the effect of strengthening magnetic pull close to the positioned magnets while reducing the over magnetic field of the magnets.

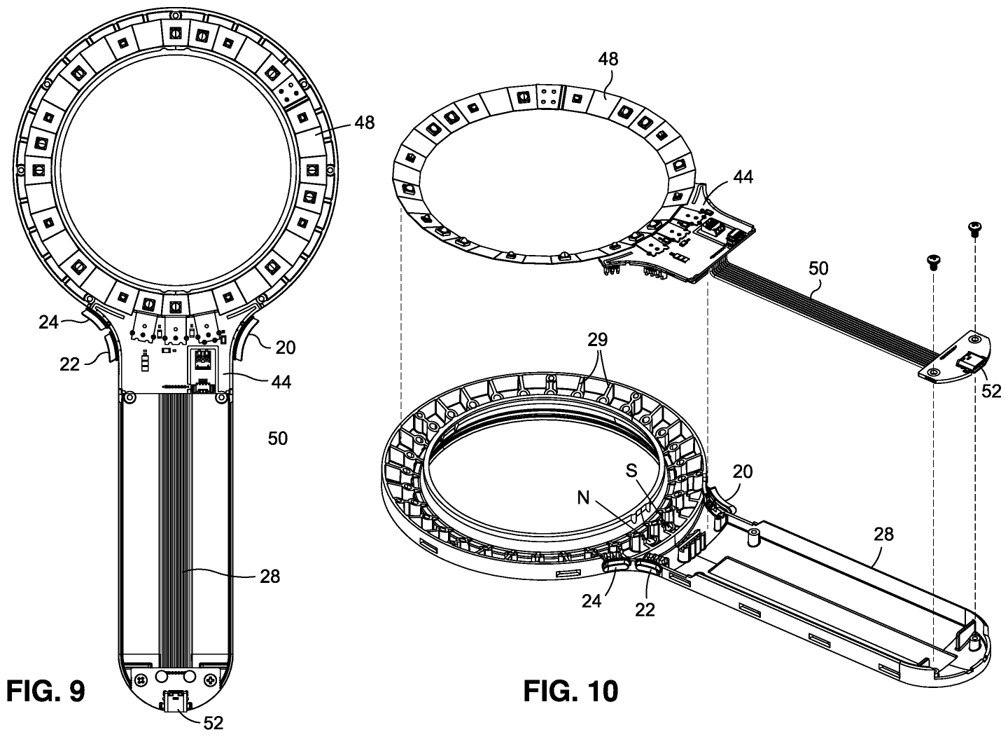

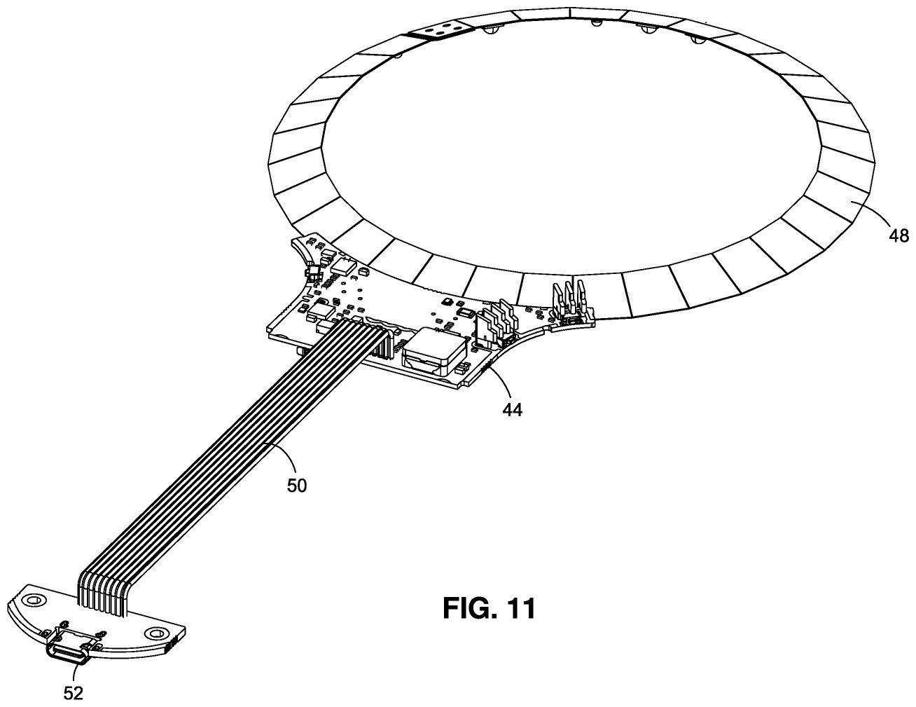

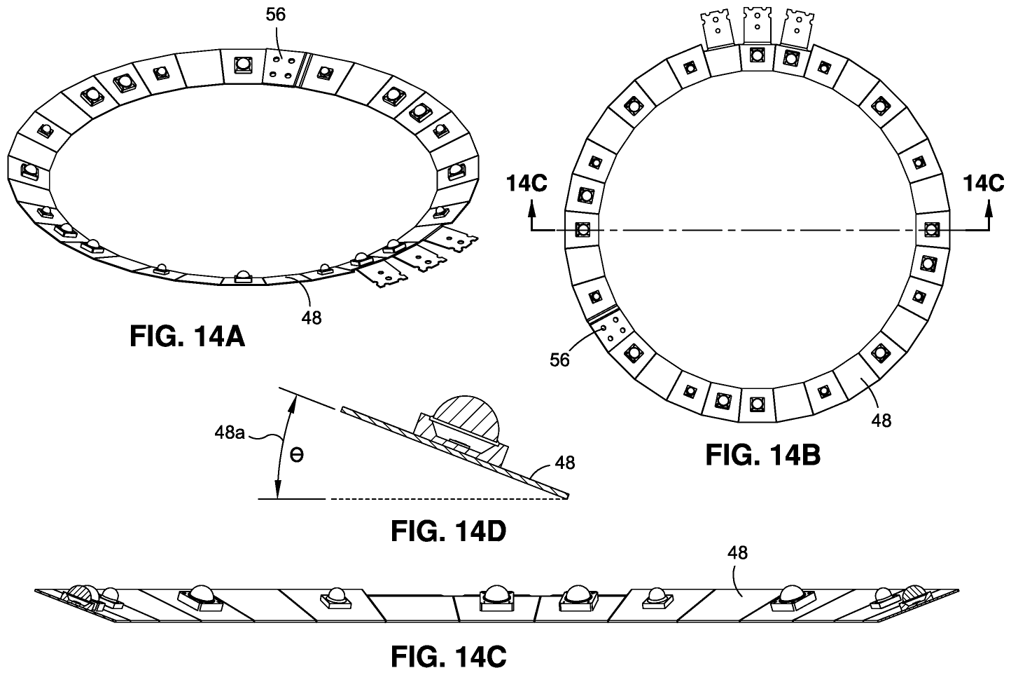

FIG. 6 shows a PCB board 44 positioned in between the upper body 28 and light separator 46. FIGS. 9 and 10 illustrate the positioning of the PBC board 44 into the body 28. The PCB board 44 comprises an annular ring PCB 48 having SMD LEDs soldered directly on the PCB 48 surface, which provides more accurate and efficient positioning of LEDs. As used herein SMD stands for Surface Mounted Devices, and in the case of SMD LEDs metallic contacts may be soldered directly to a circuit board. As discussed with regard to FIGS. 14A-14D, the PCB 48 is conically shaped in its assembled form, and nests within the upper body 28 to conform to the angled supports 29 formed around the aperture of the body 28. FIG. 11 provides a view of the reverse side of the PCB 44 and PCB 48. Leads 50 provide electrical connection between the PCB 44 and a power port 52. The power port 52 may be a USB C connection to provide quick charging for a rechargeable battery (not shown, discussed below). Although a USB C connection is disclosed for the power port 52, any suitable charging connection may be utilized. It is additionally contemplated that an induction charging system may be utilized.

The PCB 44 includes an on board microprocessor, making the device microprocessor controlled, rather than analog as with prior dermatoscopes with LED illumination. Utilization of a microprocessor results in more advanced control of the device, such as the user having the ability to use selectable lighting modes described herein, adjustable brightness discussed herein, and that upon power up, the microprocessor memory recalls the last setting and will power on the device with the illumination configuration of the last use. The microprocessor control dictates constant current through the LED drivers, so that the device has consistent brightness despite lower battery charge. A light pipe 54, shown in FIG. 6, bridges the LED indicators 26 to illuminate on the face on the of the housing 14, at 26 shown in FIG. 1. As shown in FIG. 11, the PCB 44 includes a number of electronic components that includes a microprocessor, memory, LED drivers, LED indicators, lead contacts and switch contacts.

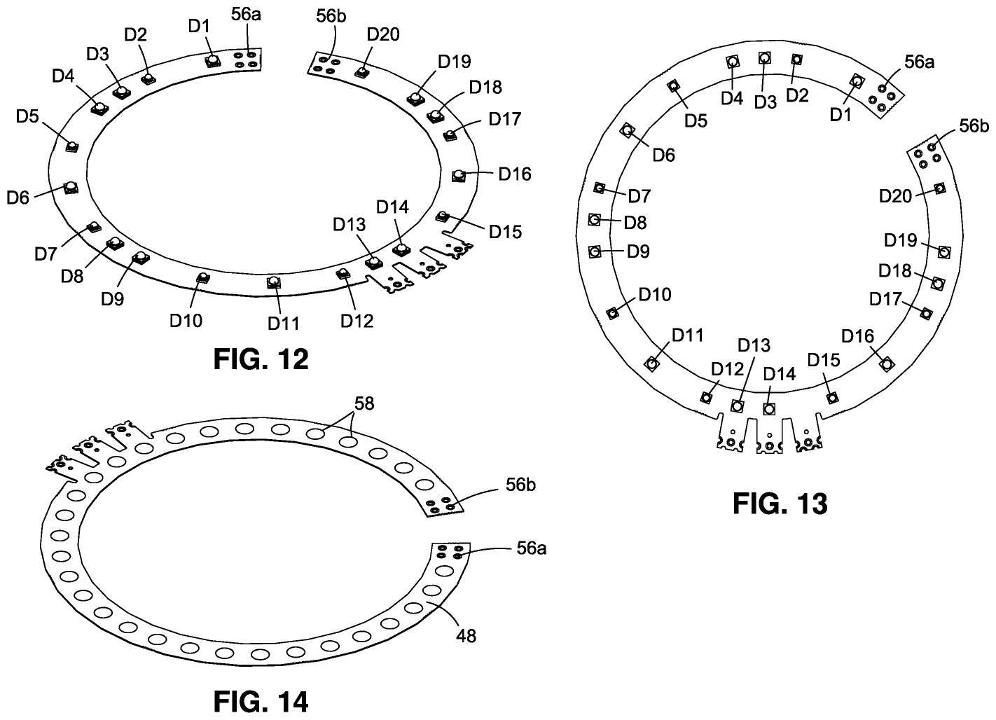

Referring to FIGS. 12-14 there is shown a perspective view, plan view and reverse side view, respectively of the annular ring PCB 48 carrying the LEDs to form the light array of the device of the present disclosure. The annular ring PCB 48 is shown in its non-assembled state, prior to be attached at ends 56a and 56b. The array comprises four different types of LEDs white light, 365 nm, 385 nm and 405 nm. As shown in FIGS. 12 and 13, the positioning and type of LEDs on the ring PCB are as follows: D1, D6, D11 and D16 are high power SMD UV LEDs 385 nm; D2, D5, D7, D10, D12, D15, D17 and D20 are high power SMD LEDs white; D3, D8, D13, D18 are high power SMD LEDs 405 nm; and D4, D9, D14 and D19 are high power SMD LEDs 365 nm. FIG. 14 shows the reverse side view of PCB 48 where copper contacts 58 that may be formed. The bare copper of the contacts 58 aid in the conduction of heat to the aluminum die cast body of the housing 14 and internal aluminum die caste components.

Referring to FIGS. 14A-14D there is shown the annular ring PCB 48 in the assembled position, wherein solder holes of 56a and 56b are aligned and interconnected at joint 56. By joining the ends 56a and 56b of the circular ring PCB 48 at joint 56, the PCB 48 forms in a conical shape as illustrated in FIG. 14A. FIG. 14B is a top plan view of the assembled PCB 48, showing axis 14C-14C. FIG. 14C shows a cross section of the PCB 48 along axis 14-C-14C showing that the LEDs are situated upon an incline, as further demonstrated in a representative FIG. 14D, showing an angle 48a relative to a plane extending at the base of cone formed by PCB 48. The angle 48a may be approximately 21.18 degrees. Although the angle is disclosed at approximately 21.18 degrees, it is contemplated that other angles may be suitable. The angled placement of the LEDs on the PCB 48 directs light to the center of the opening where magnifier 12 is situated. Likewise, the conical shape of the assembled PCB 48 is configured to nest within the body 28 and angled supports 29 (See FIG. 10).

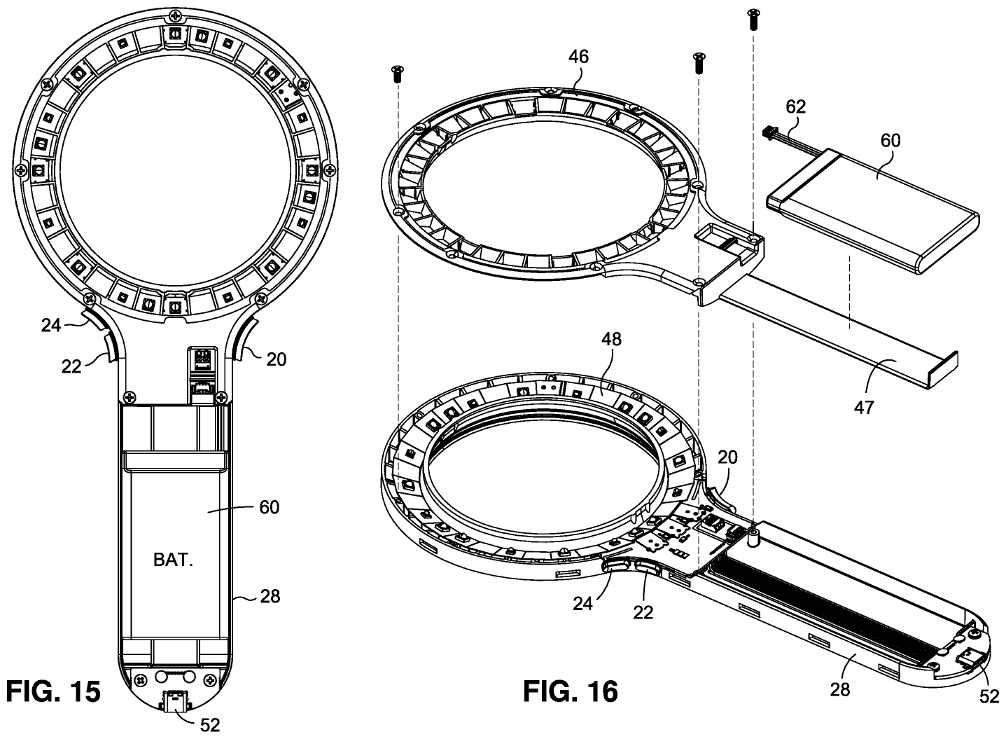

Referring to FIGS. 6, 15 and 16 there is shown a light separator 46 that is positioned over the PCB 44 and PCB 48 and provides series of chambers for each LED, to prevents light mixing between light source LEDs. The separator 46 also provides a member 47 for positioning of a battery 60 into the upper body 28, with leads interconnecting to electrical components of the PCB 44, PCB 48 and supplying power, as well as receiving a charge from charging port 52. The battery 60 is a lithium polymer battery, 3.7v. 4000 mAh, rechargeable, however it is understood that the disclosed device may utilize any suitable battery type to supply power to the device 10. The battery 60 may be affixed to the light separator 46 via adhesive 61 as shown in FIG. 6. The separator 46 may be formed of an aluminum die cast material to aid in heat dissipation.

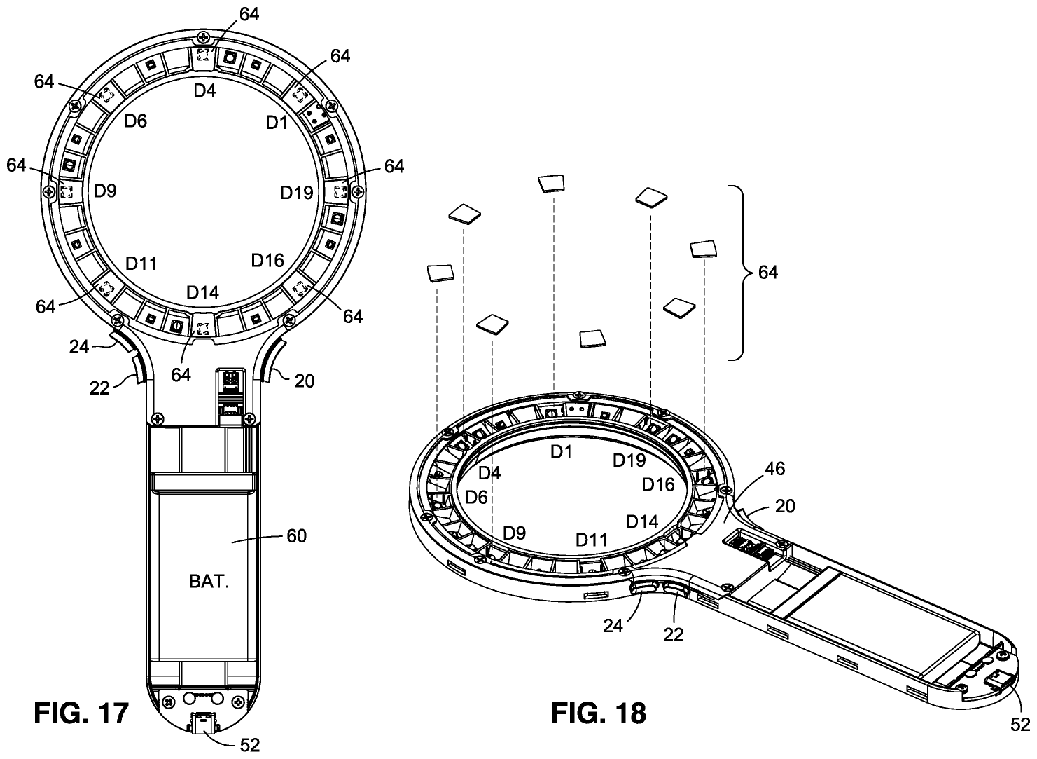

Referring to FIGS. 6, 17 and 18, there is shown the placement of ZWB2 bandpass filters over each of the 365 nm and 385 nm UV LEDs. In particular, ZWB2 bandpass filters are placed in or over the chambers of the light separator 46 formed about the following LEDs D1, D6, D11 and D16 each 385 nm UV LEDs and LEDs D4, D9, D14 and D19 each being 365 nm UV LEDs. Use of ZWB2 bandpass filters 64 result in much better contrast in image due to the elimination of visible light. As such light emitted for any of the UV LEDs of 365 nm and 385 nm will pass through the ZWB2 bandpass filters 64. The utilized bandpass filters 64 attenuates all or a significant portion of frequencies outside of a desired frequency. Because the bandpass filters 64 are placed over the indicating light transmitting LEDs a desired frequency range is transmitted through the filter, while blocking all or most of certain frequency ranges of light from passing through the filters 64. Use of ZWB2 bandpass filters result in much better contrast in an image due to the elimination of all or a significant portion of visible light. Although the disclosure herein indicates and discloses a ZWB2 filter, it is contemplated by the present disclosure that other bandpass filters may be utilized that have a similar effect of a ZWB2 namely an optical filter that has higher transmittance in the UV spectrum than the visible spectrum. The device discloses the use of a ZWB2 bandpass filter offered by Optima, Inc. of Tokyo Japan. Optima offers optical filters under the designations ZBW1, ZBW3 and ZB1 having similar characteristics as a bandpass filter, blocking some wavelengths of light and the disclosure contemplates use of such filters. In addition, other manufactures offer filters having near characteristics of the ZWB2 filter, namely Hoya Corporation of Tokyo, Japan offers U-360 and UL365 Glass filters and Schott North America, Inc of Duryea, Pa. offer a product under the designation of UG11. Hoya also offers U-340 and U-330 glass filters that may be used and other Schott products under the designations UG1 and UG5 are contemplated by the disclosure.

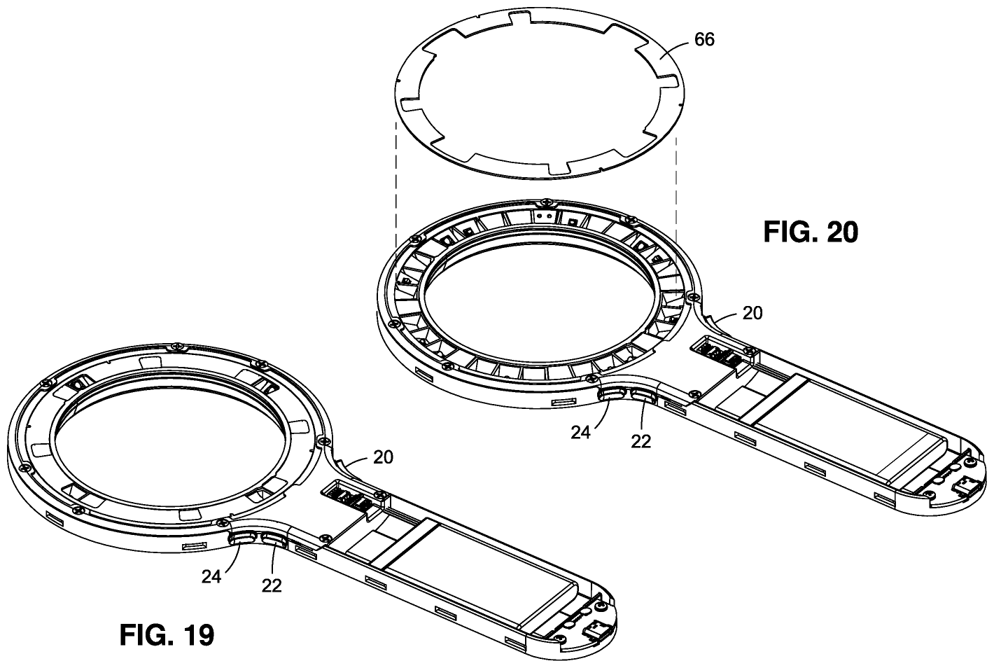

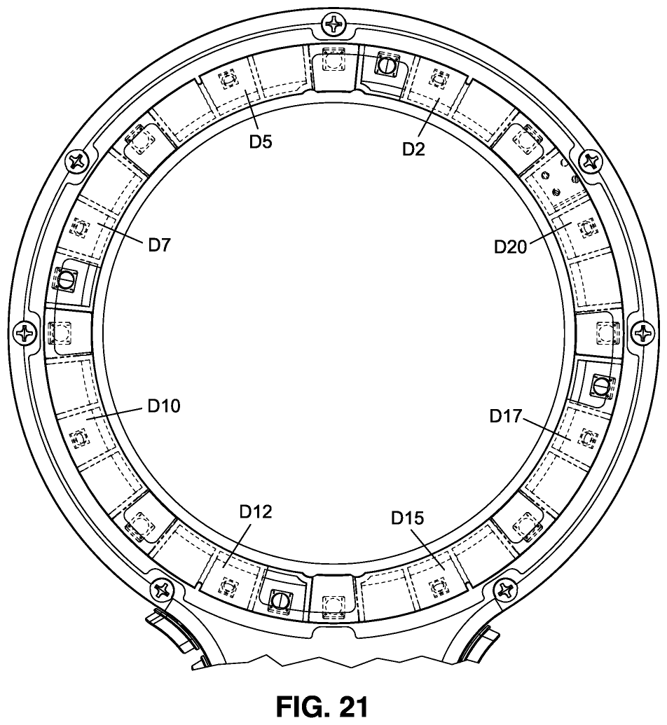

Referring to FIG. 6 there is shown a polarizer ring filter 66 positioned to attach to the light separator 46, all of which is covered by a glass disk 68 to keep the interior of the device 10 free from invading particles. The lower body 30 completes the device by interconnecting to the upper body 28 to form the housing 14. Referring to FIGS. 6, 19, 20 and 21, the polarizer ring filter 66, is positioned to cover the white LEDs D2, D5, D7, D10, D12, D15, D17 and D20. As such any light emitted from the white light LEDs D2, D5, D7, D10, D12, D15, D17 and D20 will be polarized. The Polarizer ring filter 66 axis of polarization may be 90 degrees offset from the lens filter 34. The polarizer filter 34 may be comprised of a linear polarizer film having approximately 0.030″ thickness. The positioning of the different wavelength LEDs, filters and polarizers allows for operation of the dermatoscope in at least five different modes: a first mode for polarized white LEDs; a second mode for activating only the ZWB2 bandpass filtered 365 nm UV LEDs; a third mode for activating only the ZWB2 bandpass filtered 385 nm UV LEDs; a fourth mode for activating only the 405 nm UV LEDs; and a fifth mode (may be referred to as a Woods mode) for activating only the ZWB2 bandpass filtered 365 nm UV LEDs and the ZWB2 bandpass filtered 385 nm UV LEDs. It is also contemplated by the disclosure that the ring filter 66 may have additional cut outs along the inner perimeter that may not cover certain of the white light LEDs and as such, such white light LEDs may not include an associated polarizer. As such, the disclosure contemplates a sixth mode of operation that may include activating only non-polarized white LEDs.

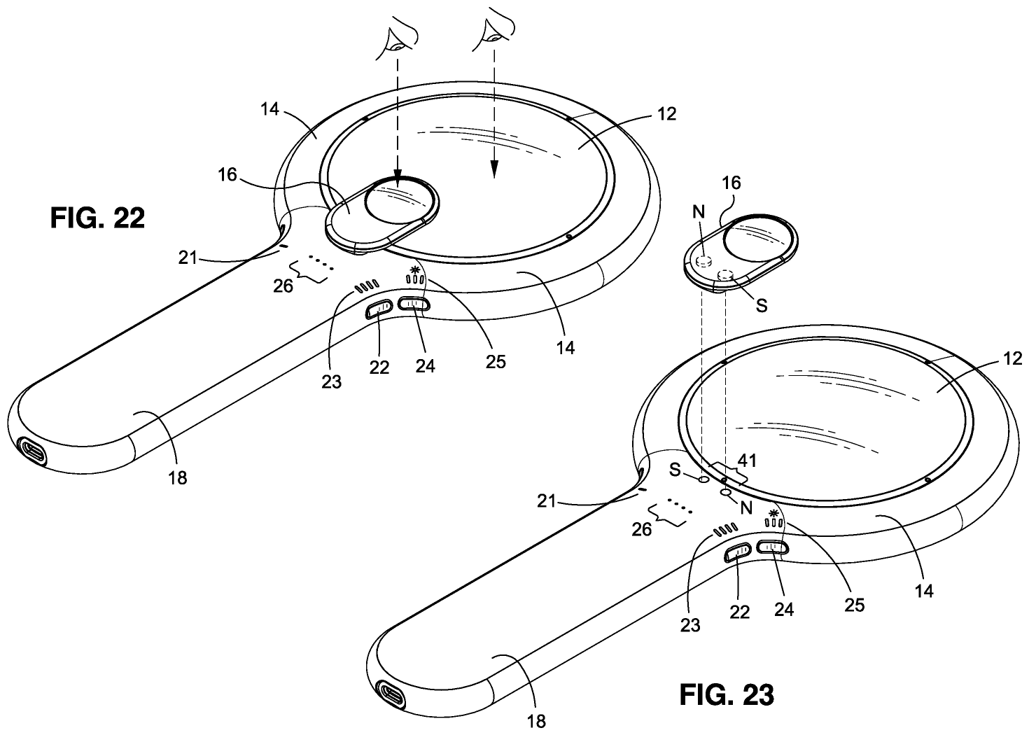

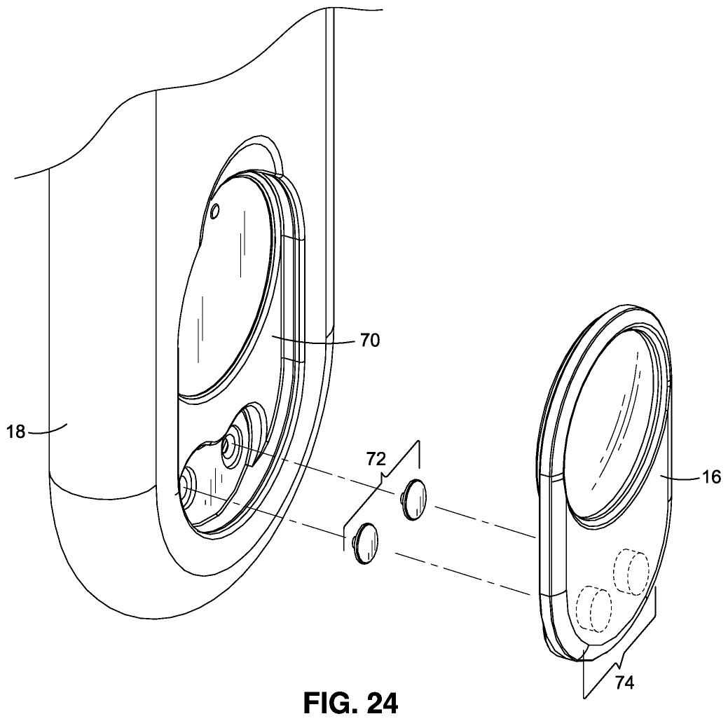

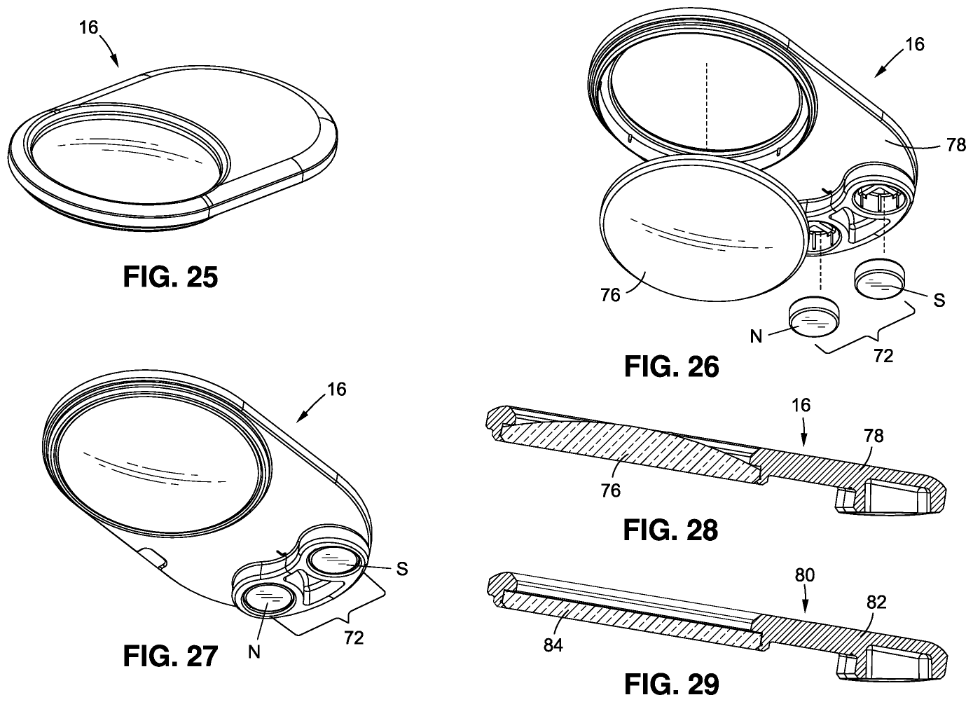

Referring particularly to FIGS. 22-28 the structure and use of auxiliary add on lens 16 is disclosed. As shown in FIG. 3, the lens 16 nests within the handle of the device 10. Once removed, the add on lens 16 can be used in the manner shown in FIGS. 22 and 23. The add on lens 16 engages magnetic points 41 on the face of the housing 14, via magnets. In addition to providing proper orientation via the N-S magnetic arrangement, magnets situated in the device 10 face 14 at magnetic points 41 are axially magnetized and placed in antiparallel relationship to each other, and corresponding magnets attached in auxiliary magnifier 16, are also axially magnetized and positioned in antiparallel relationship, and interact with the magnets of the device for placement on the device 10 at points 41. The positioning of the magnets in antiparallel arrangement reduces the long range magnetic field, as compared two magnets placed in parallel relationship. For example, magnets placed in parallel relationship may have a measured magnetic field of approximately 20 Gauss at a 25 mm distance where identical magnets placed in antiparallel relationship may have a measured magnetic field at 25 mms of twenty times less, for example 1 Gauss. Reduction of magnetic field may provide advantages in healthcare environments where it may be important to reduce magnetic fields in proximity to magnetic sensitive medical devices such as pacemakers. Magnet placement on the axillary add on lens 16 coordinates with the N-S arrangement magnetic points 41 on the face of the housing 14, so that proper orientation will be provided. Also, the strong magnetic attraction created by the antiparallel configuration of the magnets 72 and at points 14 holds the add on lens 16 in place. The addon lens 16 provides a 25 mm diameter EFL lens for high magnification view. The add on lens 16 provides an enhanced view of an area of concern without the need to switch to a different dermatoscope. FIG. 24 demonstrates the nesting feature of the add on magnifier 16, which is received into a recess 70 of the handle 18 of the device 10. The add on lens 16 is secured in place in the recess 70 via metal points 72 which are secured within the recess 70, and magnets 74 engage the metal points 72 for secure placement. FIG. 25 shows the distal side of the add on magnifier 16. FIG. 27 shows the proximal side that either engages the nesting recess 70, using magnets 72, or magnetic points 14 as shown in the face of the housing 14 as shown in FIGS. 22 and 23. FIG. 26 shows the component lens 76, body 78 and magnets 72 in exploded view. FIG. 28 represents a cross sectional view of the add on accessory 16 where the plastic body 78 is swaged by heat around the lens 76. Lens 76 has a 2.5× magnification. Also, a separate add on accessory 80 may include a magnetically attachable 25 mm diameter 495 nm bandpass filter 84 to be placed over at least a portion lens 12 to provide enhanced views of fluorescence. The construction and operation of the accessory 80, having a body 82 and bandpass filter 84 is constructed identically to the accessory 16, however, instead of having a magnification lens 76, the body 82 has a bandpass filter 84.

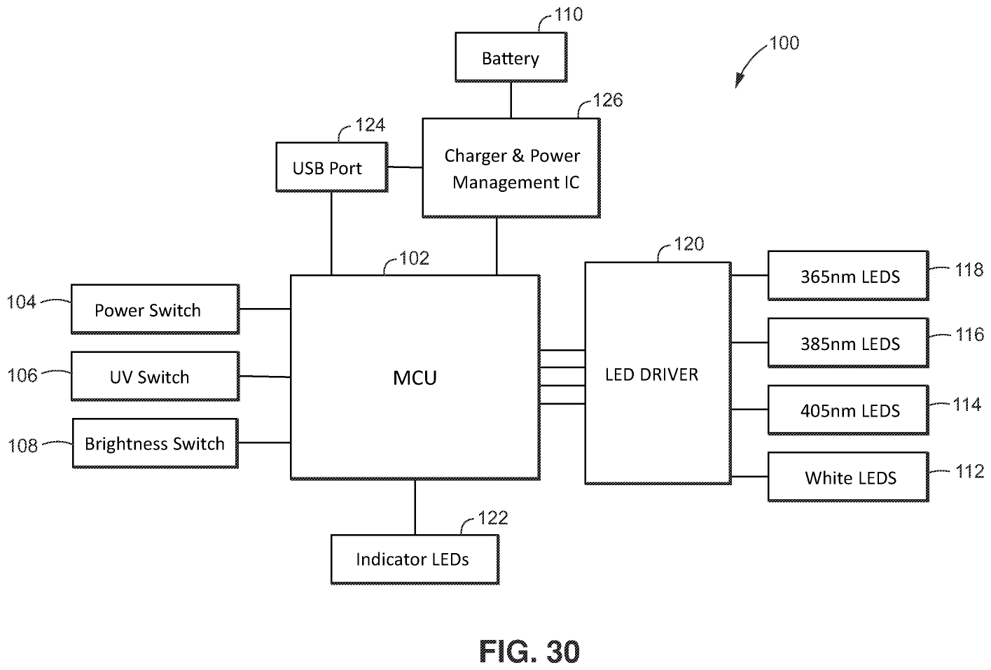

Referring to FIG. 30 there is shown a schematic of the electronic components located in or on the housing of the disclosed illumination device 100. An onboard microprocessor 102 provides operational control of the various components of the device 100. The microprocessor 102 receives signals from various user interfaces, such as a power switch 104, UV switch 106 and/or brightness switch 108. A signal from the power switch 104 initiates the power from a battery to supply power to the device 100. In operation, power switch 104 operates as follows: a single push turns from a user sends a signal to the microprocessor 102 to instruct the LED driver 118 to turn on all of the white light LEDs 112 in the LED array (eight white LEDs). A white light indicator LED is included in the group of indicator LEDs 122 which are controlled by the microprocessor 102. The indicator LEDs 122 are situated on the handle of the housing of device 100 and certain LEDs will illuminate to indicate white light mode to a user. A second push of power switch 104 sends a signal to the microprocessor to instruct the LED driver to turn on UV mode. In UV mode, the user may select UV switch 106 to select through the microprocessor 102 to instruct the LED driver 120 for activation of UV LEDS 114, 116 and 118. UV mode switch 106 activation sends a signal to the microprocessor 102 to select between four UV modes. UV mode indicator LEDs are positioned on the housing handle of the device 110 as part of indicator LEDs 122 to provide an indication to the user the particular activated UV mode. For example, the user may select between 365 nm LEDs 118 (four in the array), 385 nm LEDs 116 (four in the array), 405 nm LEDs 114 (four in the array) or Woods mode (combination of all 365 nm LEDs 118 and 385 nm LEDs 116 in the array). It is understood and contemplated that Woods mode could incorporate any other combinations of UV LEDs to best approximate the spectrum of a Woods Lamp. Brightness switch 108, allows the user to select between three levels of brightness. A signal received from the brightness switch 108 by the microprocessor instructs the LED driver 120 to activate one or combination of LEDs 112, 114, 116 and/or 118 at a predetermined intensity for all three levels of brightness. For example, a first level may be 33.3% intensity, a second level may be 66.6% intensity and a third level may be 100% intensity. A brightness level indicator LEDs are incorporated as part of the indicator LEDs 122 and provide an indication to the user of the brightness level selected. It is understood and contemplated by this disclosure that there could be any number of levels of brightness or percentages of intensity.

A USB power port 124 is provided to interconnect to the electronic components of the device 100 for supplying power directly to the microprocessor 102 and all attached electronic components or charging a battery 110 through a power management integrated circuit 126. In operation, when power is not supplied be the port 124, the chip 126 draws and directs power from the battery 110 to supply power to the microprocessor 102 and other onboard electronic components. Also, power level indicator LEDs as part of the indicator LEDs 122, act as a fuel gauge indicator with all four indicators illuminated means the batter is full or nearly full. The persistent illumination (while in use) of battery power LED indicators of the indicator LEDs 122 allows for constant monitoring of the battery level.

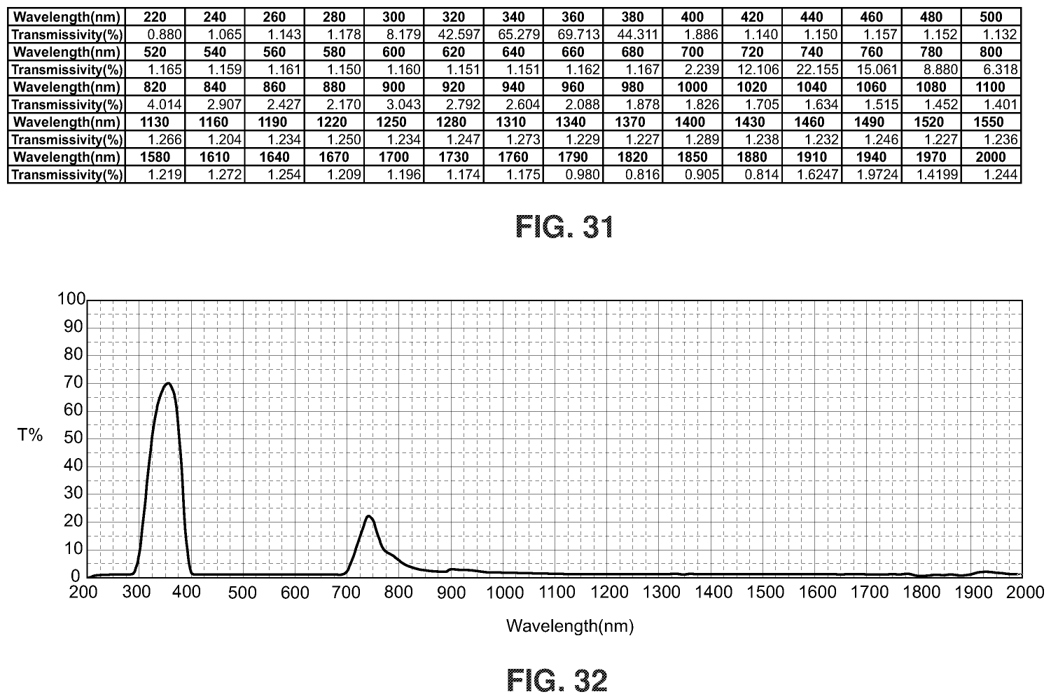

Referring particularly to FIGS. 31 and 32 there is shown a tabular chart (FIG. 31) and graphical chart 32 showing transmissivity verses wavelength information for ZWB2 filter glass bandpass filter 64, for example as shown in FIG. 18. FIGS. 31 and 32 demonstrate ZWB2 filter glass bandpass filter 64 is very effective at transmitting light in the UV-A spectrum while simultaneously blocking light in the visible spectrum. The range of light wavelengths between approximately 400 nm to 700 nm is generally considered visible by human eyes, while the range of wavelengths between 320 and 400 nm is part of the UV-A spectrum and typically not visible by human eyes. 365 nm UV-A LEDs (for example D4, D9, D14 and D19 in FIGS. 12-14) and 385 nm UV-A LEDs (for example D1, D6, D11 and D16 in FIGS. 12-14) have spectral outputs centered around 365 nm and 385 nm respectively, with spectral output tapering off above and below their specified peaks. The result is UV-A LEDs typically emit a small amount of light in the visible spectrum. When UV-A light is emitted onto certain objects those objects can emit fluorescence that is visible to the human eye. When stray visible light is present those fluorescing emissions can become washed out. A bandpass filter such as bandpass filter 64, for example, reduces stray visible light. Reducing stray visible light results in fluorescing emissions becoming more visually prominent. Therefore, using ZWB2 filter glass bandpass filter over UV-A LEDs can enhance the viewing of fluorescing features in dermoscopy. For example, a woods lamp which can be achieved using modes of operation of the present device using UV LEDs modes described herein. A woods lamp examination of the skin is typically conducted in a dark room using UV-A light by a medical practitioner to identify and view possible skin disorders. After directing the UV light to the skin, the medical practitioner will look for any fluorescence which may indicate a skin condition or skin disorder consistent with the fluorescence or other skin change. The bandpass filter(s) 64 may therefore reduce an amount of interfering visible light which may provide a medical practitioner with an enhanced or better contrasting view fluorescing features in dermoscopy.

In some embodiments, the numbers expressing dimensions, quantities, quantiles of ingredients, properties of materials, wavelengths and so forth, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclose may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the disclosure and does not pose a limitation on the scope of the claimed inventive subject matter. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed. Various objects, features, aspects and advantages of the inventive subject matter will become more apparent from the following detailed description of preferred embodiments, along with the accompanying drawing figures in which like numerals represent like components.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

The above description is given by way of example, and not limitation. Given the above disclosure, one skilled in the art could devise variations that are within the scope and spirit of the disclosure herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An illumination device for non-contact illumination of organic tissue comprising:
a hand held housing;
an optical lens incorporated into said housing for providing a magnified view of the tissue;
a lens filter for polarizing light passing through the optical lens;
at least one LED driver;
at least one activation switch;
a microprocessor for controlling the at least one LED driver and receiving signals from an activation switch;
a light source array comprising a plurality of light emitting diodes incorporated into said housing and in electrical communication with said at least one LED driver, said light source array comprising;
a plurality of white light LEDs;
a plurality of UV LEDs in the range of 365 nm-405 nm;
at least one polarizer incorporated into the housing for polarizing light emitted from at least one of the white LEDs; and
a bandpass filter incorporating into the housing for filtering light emitted from at least one of the UV LEDs.

2. The illumination device of claim 1 wherein said the microprocessor is adapted to selectively transition the light source array between modes of operation, said modes of operation comprising:
a first mode for activating the at least one polarized white light;
a second mode for activating the at least one bandpass filtered UV LED; and
a third mode for activating at least one of the plurality of UV LEDs without filtering.

3. The illumination device of claim 1 wherein said plurality of UV LEDs further comprise;
at least one bandpass filtered 365 nm UV LED;
at least one bandpass filtered 385 nm UV LED; and
at least one 405 nm UV LED

4. The illumination device of claim 3 wherein said the microprocessor is adapted to selectively transition the light source array between modes of operation, said modes of operation comprising:
a first mode for activating only the at least one bandpass filtered 365 nm UV LED;
a second mode for activating only the at least one bandpass filtered 385 nm UV LED;
a third mode for activating only the at least one 405 nm UV LED; and
a fourth mode for activating only the at least one bandpass filtered 365 nm UV LED and the at least one bandpass filtered 385 nm UV LED.

5. The device of claim 4 wherein said modes of operation further comprises a fifth mode of operation for activating only the at least one polarized white light.

6. The device of claim 1 wherein the bandpass filter is a ZWB2 band pass filter.

7. The device of claim 1 wherein the bandpass filter is a UV bandpass filter.

8. The device of claim 1 wherein the bandpass filter that transmits UV light and filters visible light.

9. The device of claim 1 wherein the bandpass filter is an optical filter that has higher transmittance in the UV spectrum than the visible spectrum.

10. The illumination device of claim 1 further comprising a battery power source incorporated into said housing.

11. The illumination device of claim 1 wherein the lens magnification is in the range of 2× to 3×.

12. The illumination device of claim 1 wherein the lens magnification is 2.3×.

13. The illumination device of claim 1 wherein the lens comprises aspheric lens surfaces.

14. The illumination device of claim 1 wherein the LEDs and UV LEDs are high power SMD LEDs.

15. The illumination device of claim 1 wherein the plurality of white LEDs comprise eight white LEDs.

16. The illumination device of claim 1 wherein the plurality of UV LEDs comprise 4 V LEDs 365 nm, 4 UV LEDs 385 nm and 4 UV LEDs 405 nm.

17. The illumination device of claim 1 wherein a recess formed in the hand held housing receives a selectively removable secondary magnifier.

18. The illumination device of claim 13 wherein the secondary magnifier has 2.5× magnification.

19. The illumination device of claim 1 wherein a secondary magnifier is positionable over optical lens via magnetic attachment to the hand held housing.

20. The illumination device of claim 15 wherein the magnetic attachment of the secondary magnifier includes at least two axially magnetized magnets placed in antiparallel arrangement.

21. The illumination device of claim 15 wherein the magnetic attachment of the hand held housing includes at least two axially magnetized magnets placed in antiparallel arrangement.

22. The illumination device of claim 2 wherein a fourth mode of operation for activating at least one non-polarized white light.

23. The illumination device of claim 4 wherein said modes of operation further comprises a fifth mode of operation for activating only at least one non-polarized white light\

24. The illumination device of claim 1 wherein said plurality of light emitting diodes are attached to PCB, wherein at least a portion of the PCB is formed in a conical configuration.

25. An illumination device for non-contact illumination of organic tissue comprising:
a hand held housing;
an optical lens incorporated into said housing for providing a magnified view of the tissue;
at least one UV LED in the range of 365 nm to 405 nm;
a bandpass filter incorporated into the housing for filtering visible light from said at least one of the UV LEDs.

26. The device of claim 25 wherein the bandpass filter is a ZWB2 band pass filter.

27. The device of claim 25 wherein the bandpass filter is a UV bandpass filter.

28. The device of claim 25 wherein the bandpass filter that transmits UV light and filters visible light.

29. The device of claim 25 wherein the bandpass filter is an optical filter that has higher transmittance in the UV spectrum than the visible spectrum.
